(19) United States
(12) Patent Application Publication
(10) Pub. No.: US 2024/0392242 A1
(43) Pub. Date: Nov. 28, 2024

(54) METHOD FOR EXTRACTING EPIDERMAL CELLS

(71) Applicant: Hangzhou Singclean Medical Products Co., Ltd, Hangzhou (CN)

(72) Inventors: Qiulin He, Hangzhou (CN); Yaru Wu, Hangzhou (CN); Jingwei Zhang, Hangzhou (CN); Zhiwei Nie, Hangzhou (CN); Haiyi Hu, Hangzhou (CN); Wei Huang, Hangzhou (CN)

(21) Appl. No.: 18/228,016

(22) Filed: Jul. 31, 2023

(30) Foreign Application Priority Data
May 25, 2023 (CN) 202310597186.2

(51) Int. Cl. C12N 5/071; C12N 9/64; C12N 9/76

(52) U.S. Cl. CPC C12N 5/0629; C12N 9/6424; C12N 9/6427; C12N 9/6491; C12Y 304/21004; C12Y 304/21064; C12Y 304/24007; C12N 2509/00

(57) ABSTRACT

The present disclosure discloses a method for extracting epidermal cells. The method includes soaking skin tissues in a digestive juice, and obtaining an epidermal cell suspension through alternate use of a high-temperature digestion condition and a low-temperature digestion condition. The method can be used for clinical research or treatment of burns, scalds, pigmentation and hypopigmentation, ulcers, aesthetic dermatology and the like. Through adjusting the digestion conditions of the skin tissues, the method separate the epidermis from the dermis within a relatively short time, and have the advantages of extracting epidermal cells in large amount, high survival rate and high activity. The extracted epidermal cells are applicable to various clinical indications.

### CROSS-REFERENCE TO RELATED APPLICATION

The present application claims priority from Chinese Patent Application No. 202310597186.2 filed on May 25, 2023, the contents of which are incorporated herein by reference in their entirety.

### TECHNICAL FIELD

The present disclosure relates to a cell extraction technology, and in particular, to a method for extracting epidermal cells.

### BACKGROUND

Epidermal cells, as one of the main cell types in human skin, play an important role in maintaining the skin barrier function, and participating in immune response and wound healing, etc. Therefore, the technologies for extracting and culturing epidermal cells have extensive application and research value in the fields of medicine and life sciences.

At present, the commonly used technologies for extracting epidermal cells include an enzymatic digestion method, a mechanical separation method, an ultrasonic method, etc. Among them, the enzymatic digestion method is the most commonly used method in clinical treatment, involving the following patents: CN104087551B, CN103623001B, CN103614295B, and CN111849871A. In each of the patents disclosed above, epidermal tissues are digested at a constant temperature, which has the disadvantages of (1) longer digestion time, or (2) less extracted epidermal cells and lower survival rate and cell activity.

To this end, the present disclosure provides a novel technology for extracting epidermal cells, which extracts an epidermal cell suspension with large extraction amount, high survival rate and high activity within a relatively short time by combining high-temperature digestion conditions with low-temperature digestion conditions.

### SUMMARY

In view of the shortcomings of the prior art, the present disclosure improves the prior art and provides a method for extracting epidermal cells, which is realized by the following technical solution:

The present disclosure discloses a novel method for extracting epidermal cells, including: soaking skin tissues in a digestive juice, and obtaining an epidermal cell suspension through alternate use of a high-temperature digestion condition and a low-temperature digestion condition. The method specifically includes the following steps:
1) taking out the skin tissues in a sterile environment, putting the skin tissues into a culture dish, and washing the skin tissues with normal saline;
2) soaking epidermal tissues in the digestive juice to perform digestion, where a digestion environment in the digestive juice involves alternation of the high-temperature digestion condition and the low-temperature digestion condition or the low-temperature digestion condition and the high-temperature digestion condition;
3) after digestion, separating epidermis from dermis; and
4) resuspending the epidermal tissues with normal saline to, performing counting and determining a cell survival rate;
wherein the method is capable of obtaining the epidermal cell suspension within 4 h, and in the cell suspension, a cell extraction amount is greater than 1×10⁷ cells/10 cm², and the cell survival rate is higher than 85%.

As a further improvement, according to the present disclosure, step 2) specifically includes: soaking the skin tissues in the digestive juice, performing digestion at 25° C. to 40° C. for 0.1 h to 2 h and then at 0° C. to 10° C. for 0.1 h to 2 h, and then performing digestion under the above conditions alternately; or soaking the skin tissues in the digestive juice, and performing digestion at 0° C. to 10° C. for 0.1 h to 8 h and then at 25° C. to 40° C. for 0.1 h to 8 h.

As a further improvement, according to the present disclosure, a total time range for the high-temperature digestion condition is 0.1 h to 8 h, and a total time range for the low-temperature digestion condition is 0.1 h to 8 h.

As a further improvement, according to the present disclosure, the skin tissues are derived from a human or an animal.

As a further improvement, according to the present disclosure, the digestive juice is any one, two or three of collagenase, dispase, a TrypLE™ cell dissociation agent, trypsin, ethylenediamine tetraacetic acid, and an Accutase™ cell dissociation agent.

As a further improvement, according to the present disclosure, a concentration range of the collagenase is 50 U/mL to 1000 U/mL, a concentration range of the dispase is 0.01 U/mL to 10 U/mL, a concentration range of the TrypLE™ cell dissociation agent is 0.01× to 10×, a concentration range of the trypsin is 0.01% to 10%, a concentration range of the ethylenediamine tetraacetic acid is 0.01% to 10%, and a concentration range of the Accutase™ cell dissociation agent is 0.01× to 10×.

As a further improvement, according to the present disclosure, the epidermal cells are used for clinical research or treatment of burns, scalds, pigmentation and hypopigmentation, ulcers, aesthetic dermatology and the like.

The present disclosure provides a technology for extracting epidermal cells based on alteration of cell digestion conditions, which may be used for clinical research or treatment of burns, scalds, pigmentation and hypopigmentation, ulcers, aesthetic dermatology and the like. According to the method, the digestion conditions of the skin tissues are adjusted, so that the epidermis is separated from the dermis within a relatively short time, and the epidermal cells have the advantages of large extraction amount, high survival rate and high activity. The extracted epidermal cells are applicable to various clinical indications.

The present disclosure has the following beneficial effects:
1. By alternately using the high-temperature digestion condition and the low-temperature digestion condition, the cell extraction time is shortened, and the cell extraction amount, survival rate and activity are improved. Compared with constant-temperature digestion at low temperature, the digestion at alternate high temperature and low temperature can shorten the digestion time by 2 to 3 times (from more than 10 h to less than 4 h). Compared with constant-temperature digestion at low temperature and constant-temperature digestion at high temperature, the digestion at alternate high temperature and low temperature increases the cell extraction amount by 4 to 20 times (from less than 5.0×10⁶ cells/10 cm² to more than 2.5×10⁷ cells/10 cm²), increases the cell survival rate by 1.13 to 1.21 times (from less than 75% to more than 85%), and increases the cell activity by 1.13 to 1.29 times (from less than 0.85 to more than 0.90).
2. By controlling the high temperature to range from 25° C. to 40° C. and the low temperature to range from 0° C. to 10° C., the cell damage is reduced, and the cell extraction amount, survival rate and activity are improved. Compared with the high temperature above 40° C. or below 25° C. and the low temperature above 10° C. or below 0° C., the condition of the high temperature ranging from 25° C. to 40° C. and the low temperature ranging from 0° C. to 10° C. increases the cell extraction amount by 10 to 15 times (from less than 4.0×10⁶ cells/10 cm² to more than 3.0×10⁷ cells/10 cm²), increases the cell survival rate by 1.13 to 1.31 times (from less than 75% to more than 85%), and increases the cell activity by 1.22 to 1.69 times (from less than 0.85 to more than 1.00).
3. By controlling the high-temperature digestion time to range from 0.1 h to 8 h and the low-temperature digestion time to range from 0.1 h to 8 h, the cell damage is reduced, and the cell extraction amount, survival rate and activity are improved. Compared with the high-temperature digestion time of more than 8 h or less than 0.1 h and the low-temperature digestion time of more than 8 h or less than 0.1 h, the condition of the high-temperature digestion time ranging from 0.1 h to 8 h and the low-temperature digestion time ranging from 0.1 h to 8 h increases the cell extraction amount by 10 to 15 times (from less than 4.2×10⁶ cells/10 cm² to more than 3.2×10⁷ cells/10 cm²), increases the cell survival rate by 1.13 to 1.31 times (from less than 75% to more than 85%), and increases the cell activity by 1.22 to 1.69 times (from less than 0.85 to more than 1.10).

### DETAILED DESCRIPTION

The technical solution of the present disclosure will be further explained by specific examples below:

### Example 1

This example is specifically implemented as follows:
1. Epidermal tissues from a human were taken out in a sterile environment, put into a culture dish, and washed with normal saline.
2. The epidermal tissues were equally divided into 4 groups, and the surface area of each group was 10 cm²:
group 1: soaked in a digestive juice composed of 100 U/mL collagenase and 1 U/mL dispase, digested at 10° C. for 2 h, and then transferred to be subjected to digestion at room temperature for 2 h;
group 2: soaked in a digestive juice composed of 100 U/mL collagenase and 1 U/ml dispase, digested at room temperature for 2 h, and then transferred to be subjected to digestion at 10° C. for 2 h;
group 3: soaked in a digestive juice composed of 100 U/mL collagenase and 1 U/mL dispase, and digested at 10° C. for 4 h; and
group 4: soaked in a digestive juice composed of 100 U/mL collagenase and 1 U/mL dispase, and digested at room temperature for 4 h.
3. After digestion, epidermis was separated from dermis.
4. After the epidermal tissues were resuspended with normal saline, counting was performed, and a cell survival rate was determined.
5. The cells were inoculated and cultured for 24 h, and a growth activity was determined by an MTT method.

Implementation results are shown below:

| | Cell | Cell | Cell |
|---|---|---|---|
| | extraction | survival | activ- |
| Group | amount | rate | ity |
| Group 1: digested at 10° C. for 2 h, | 2.56 × 10⁷ cells | 88.66% | 0.91 |
| and digested at room temperature | | | |
| for 2 h | | | |
| Group 2: digested at room | 2.93 × 10⁷ cells | 86.72% | 0.90 |
| temperature for 2 h, and digested at | | | |
| 10° C. for 2 h | | | |
| Group 3: digested at 10° C. for 4 h | 1.33 × 10⁶ cells | 74.91% | 0.82 |
| Group 4: digested at room | 4.33 × 10⁶ cells | 70.91% | 0.71 |
| temperature for 4 h | | | |

The above results of this example show that within the same digestion time, compared with group 3 (constant-temperature digestion at low temperature) and group 4 (constant-temperature digestion at high temperature), group 1 (digestion at alternate low temperature and high temperature) and group 2 (digestion at alternate high temperature and low temperature) significantly increase the cell extraction amount, survival rate and activity. Compared with constant-temperature digestion at low temperature and constant-temperature digestion at high temperature, the digestion at alternate high temperature and low temperature increases the cell extraction amount by 4 to 20 times (from less than 5.0×10⁶ cells/10 cm² to more than 2.5×10⁷ cells/10 cm²), increases the cell survival rate by 1.13 to 1.21 times (from less than 75% to more than 85%), and increases the cell activity by 1.13 to 1.29 times (from less than 0.85 to more than 0.90). The results of this example indicate that the cell extraction amount, survival rate and activity can be improved by alternately using high temperature and low temperature.

### Example 2

This example is specifically implemented as follows:
1. Epidermal tissues from a human were taken out in a sterile environment, put into a culture dish, and washed with normal saline.
2. The epidermal tissues were equally divided into 3 groups, and the surface area of each group was 10 cm²:
group 1: soaked in a digestive juice composed of 100 U/mL collagenase and 1 U/mL dispase, digested at 10° C. for 2 h, and then transferred to be subjected to digestion at room temperature;
group 2: soaked in a digestive juice composed of 100 U/mL collagenase and 1 U/mL dispase, digested at room temperature for 2 h, and then transferred to be subjected to digestion at 10° C.; and
group 3: soaked in a digestive juice composed of 100 U/mL collagenase and 1 U/mL dispase, and digested at 10° C.
3. At 4, 6, 8, 10 and 12 h of digestion, whether the tissues had been completely digested was observed, and the digestion was stopped after complete digestion.

Implementation results are shown below:

| | Digestion time (h) | | | | |
|---|---|---|---|---|---|
| Condition | 4 | 6 | 8 | 10 | 12 |
| Group 1: digested at | Almost | Completely | / | / | / |
| 10° C. for 2 h, and then | completely | digested | | | |
| transferred to be | digested | | | | |
| subjected to digestion at | | | | | |
| room temperature | | | | | |
| Group 2: digested at | Completely | / | / | / | / |
| room temperature for 2 | digested | | | | |
| h, and then transferred | | | | | |
| to be subjected to | | | | | |
| digestion at 10° C. | | | | | |
| Group 3: digested at | Incompletely | Incompletely | Incompletely | Incompletely | Completely |
| 10° C. | digested | digested | digested | digested | digested |

The results of the above example show that the digestion time of group 1 (digestion at alternate low temperature and high temperature) and group 2 (digestion at alternate high temperature and low temperature) is shorter than that of group 3 (constant-temperature digestion at low temperature). Compared with constant-temperature digestion at low temperature, the digestion at alternate high temperature and low temperature can shorten the digestion time by 2 to 3 times (from more than 10 h to less than 4 h). The results of this example indicate that the extraction time can be shortened by alternately using high temperature and low temperature.

### Example 3

This example is specifically implemented as follows:
1. Epidermal tissues from the back of a mouse were taken out in a sterile environment, put into a culture dish, and washed with normal saline.
2. The epidermal tissues were equally divided into 4 groups, and the surface area of each group was 10 cm²:
group 1: soaked in a digestive juice composed of 1×TrypLE™ cell dissociation agent and 1 U/mL dispase, digested at 10° C. for 2 h, and then transferred to be subjected to digestion at 37° C. for 2 h;
group 2: soaked in a digestive juice composed of 1×TrypLE™ cell dissociation agent and 1 U/mL dispase, digested at 37° C. for 2 h, and then transferred to be subjected to digestion at 10° C. for 2 h;
group 3: soaked in a digestive juice composed of 1×TrypLE™ cell dissociation agent and 1 U/mL dispase, digested at −10° C. for 2 h, and then transferred to be subjected to digestion at 37° C. for 2 h;
group 4: soaked in a digestive juice composed of 1×TrypLE™ cell dissociation agent and 1 U/mL dispase, digested at 37° C. for 2 h, and then transferred to be subjected to digestion at −10° C. for 2 h;
group 5: soaked in a digestive juice composed of 1×TrypLE™ cell dissociation agent and 1 U/mL dispase, digested at 10° C. for 2 h, and then transferred to be subjected to digestion at 45° C. for 2 h;
group 6: soaked in a digestive juice composed of 1×TrypLE™ cell dissociation agent and 1 U/mL dispase, digested at 45° C. for 2 h, and then transferred to be subjected to digestion at 10° C. for 2 h;
3. After digestion, epidermis was separated from dermis.
4. After the epidermal tissues were resuspended with normal saline, counting was performed, and a cell survival rate was determined.
5. The cells were inoculated and cultured for 24 h, and a growth activity was determined by an MTT method.

Implementation results are shown below:

| | Cell | Cell | Cell |
|---|---|---|---|
| | extraction | survival | activ- |
| Group | amount | rate | ity |
| Group 1: digested at 10° C. for 2 h, | 3.06 × 10⁷ cells | 88.20% | 1.32 |
| and digested at 37° C. for 2 h | | | |
| Group 2: digested at 37° C. for 2 h, | 3.53 × 10⁷ cells | 85.34% | 1.02 |
| and digested at 10° C. for 2 h | | | |
| Group 3: digested at −10° C. for 2 | 2.28 × 10⁶ cells | 73.43% | 0.83 |
| h, and digested at 37° C. for 2 h | | | |
| Group 4: digested at 37° C. for 2 h, | 2.34 × 10⁶ cells | 74.26% | 0.82 |
| and digested at −10° C. for 2 h | | | |
| Group 5: digested at 10° C. for 2 h, | 3.36 × 10⁶ cells | 63.30% | 0.68 |
| and digested at 45° C. for 2 h | | | |
| Group 6: digested at 45° C. for 2 h, | 3.65 × 10⁶ cells | 65.31% | 0.66 |
| and digested at 10° C. for 2 h | | | |

The results of above example show that within the same digestion time, compared with group 3 (the low temperature being −10° C. and the high temperature being 37° C.), group 4 (the low temperature being −10° C. and the high temperature being 37° C.), group 5 (the low temperature being 10° C. and the high temperature being 45° C.) and group 6 (the low temperature being 10° C. and the high temperature being 45° C.), group 1 (the low temperature being 10° C. and the high temperature being 37° C.) and group 2 (the low temperature being 10° C. and the high temperature being 37° C.) significantly increase the cell extraction amount, survival rate and activity. Compared with the high temperature above 40° C. and the low temperature below 0° C., the condition of the high temperature ranging from 25° C. to 40° C. and the low temperature ranging from 0° C. to 10° C. increases the cell extraction amount by 10 to 15 times (from less than 4.0×10⁶ cells/10 cm² to more than 3.0×10⁷ cells/10 cm²), increases the cell survival rate by 1.13 to 1.31 times (from less than 75% to more than 85%), and increases the cell activity by 1.22 to 1.69 times (from less than 0.85 to more than 1.00). The results of this example indicate that the cell damage can be reduced and the cell extraction amount, survival rate and activity can be improved by controlling the temperature ranges of the high temperature and the low temperature.

### Example 4

This example is specifically implemented as follows:
1. Epidermal tissues from the back of a mouse were taken out in a sterile environment, put into a culture dish, and washed with normal saline.
2. The epidermal tissues were equally divided into 6 groups:
group 1: soaked in a digestive juice composed of 1×TrypLE™ cell dissociation agent and 1 U/mL dispase, digested at 10° C. for 2 h, and then transferred to be subjected to digestion at 37° C. for 2 h;
group 2: soaked in a digestive juice composed of 1×TrypLE™ cell dissociation agent and 1 U/mL dispase, digested at 37° C. for 2 h, and then transferred to be subjected to digestion at 10° C. for 2 h;
group 3: soaked in a digestive juice composed of 1×TrypLE™ cell dissociation agent and 1 U/mL dispase, digested at 10° C. for 2 h, and then transferred to be subjected to digestion at 37° C. for 10 h;
group 4: soaked in a digestive juice composed of 1×TrypLE™ cell dissociation agent and 1 U/mL dispase, digested at 37° C. for 10 h, and then transferred to be subjected to digestion at 10° C. for 2 h;
group 5: soaked in a digestive juice composed of 1×TrypLE™ cell dissociation agent and 1 U/mL dispase, digested at 10° C. for 10 h, and then transferred to be subjected to digestion at 37° C. for 2 h;
group 6: soaked in a digestive juice composed of 1×TrypLE™ cell dissociation agent and 1 U/mL dispase, digested at 37° C. for 2 h, and then transferred to be subjected to digestion at 10° C. for 10 h;
3. After digestion, epidermis was separated from dermis.
4. After the epidermal tissues were resuspended with normal saline, counting was performed, and a cell survival rate was determined.
5. The cells were inoculated and cultured for 24 h, and a growth activity was determined by an MTT method.

Implementation results are shown below:

| | Cell | Cell | Cell |
|---|---|---|---|
| | extraction | survival | activ- |
| Group | amount | rate | ity |
| Group 1: digested at 10° C. for 2 h, | 3.23 × 10⁷ cells | 85.20% | 1.21 |
| and digested at 37° C. for 2 h | | | |
| Group 2: digested at 37° C. for 2 h, | 3.66 × 10⁷ cells | 86.22% | 1.10 |
| and digested at 10° C. for 2 h | | | |
| Group 3: digested at 10° C. for 2 h, | 4.36 × 10⁶ cells | 66.31% | 0.71 |
| and digested at 37° C. for 10 h | | | |
| Group 4: digested at 37° C. for 10 | 4.22 × 10⁶ cells | 63.28% | 0.63 |
| h, and digested at 10° C. for 2 h | | | |
| Group 5: digested at 10° C. for 10 | 3.86 × 10⁶ cells | 70.36% | 0.83 |
| h, and digested at 37° C. for 2 h | | | |
| Group 6: digested at 37° C. for 2 h, | 3.55 × 10⁶ cells | 73.67% | 0.84 |
| and digested at 10° C. for 10 h | | | |

The results of above example show that within the same digestion time, compared with group 3 (at low temperature for 2 hours and high temperature for 10 hours), group 4 (at low temperature for 2 hours and high temperature for 10 hours), group 5 (at low temperature for 10 hours and high temperature for 2 hours) and group 6 (at low temperature for 10 hours and high temperature for 2 hours), group 1 (at low temperature for 2 h and high temperature for 2 h) and group 2 (at low temperature for 2 h and high temperature for 2 h) significantly increase the cell extraction amount, survival rate and activity. Compared with the high-temperature digestion time of more than 8 h and the low-temperature digestion time of more than 8 h, the condition of the high-temperature digestion time ranging from 0.1 h to 8 h and the low-temperature digestion time ranging from 0.1 h to 8 h increases the cell extraction amount by 10 to 15 times (from less than 4.2×10⁶ cells/10 cm² to more than 3.2×10⁷ cells/10 cm²), increases the cell survival rate by 1.13 to 1.31 times (from less than 75% to more than 85%), and increases the cell activity by 1.22 to 1.69 times (from less than 0.85 to more than 1.10). The results of this example indicate that the cell damage can be reduced and the cell extraction amount, survival rate and activity can be improved by controlling the time ranges of the high-temperature digestion and the low-temperature digestion.

What is claimed is:

1. A method for extracting epidermal cells, comprising: soaking skin tissues in a digestive juice, and obtaining an epidermal cell suspension through alternate use of a high-temperature digestion condition and a low-temperature digestion condition, the method specifically comprising the following steps:
1) taking out the skin tissues in a sterile environment, putting the skin tissues into a culture dish, and washing the skin tissues with normal saline;
2) soaking epidermal tissues in the digestive juice to perform digestion, wherein a digestion environment in the digestive juice involves alternation of the high-temperature digestion condition and the low-temperature digestion condition or the low-temperature digestion condition and the high-temperature digestion condition;
3) after digestion, separating epidermis from dermis; and
4) resuspending the epidermal tissues with normal saline to obtain the epidermal cell suspension;
wherein the method is capable of obtaining the epidermal cell suspension within 4 h, and in the cell suspension, a cell extraction amount is greater than 1×10⁷ cells/10 cm², and the cell survival rate is higher than 85%.

2. The method for extracting epidermal cells according to claim 1, wherein step 2) specifically comprises: soaking the skin tissues in the digestive juice, performing digestion at 25° C. to 40° C. for 0.1 h to 2 h and then at 0° C. to 10° C. for 0.1 h to 2 h, and then performing digestion under the above conditions alternately; or soaking the skin tissues in the digestive juice, and performing digestion at 0° C. to 10° C. for 0.1 h to 8 h and then at 25° C. to 40° C. for 0.1 h to 8 h.

3. The method for extracting epidermal cells according to claim 1, wherein a total time range for the high-temperature digestion condition is 0.1 h to 8 h, and a total time range for the low-temperature digestion condition is 0.1 h to 8 h.

4. The method for extracting epidermal cells according to claim 3, wherein the skin tissues are derived from a human or an animal.

5. The method for extracting epidermal cells according to claim 1, wherein the digestive juice is any one, two or three of collagenase, dispase, a TrypLE™ cell dissociation agent, trypsin, ethylenediamine tetraacetic acid, and an Accutase™ cell dissociation agent.

6. The method for extracting epidermal cells according to claim 5, wherein a concentration range of the collagenase is 50 U/mL to 1000 U/mL, a concentration range of the dispase is 0.01 U/mL to 10 U/mL, a concentration range of the TrypLE™ cell dissociation agent is 0.01× to 10×, a concentration range of the trypsin is 0.01% to 10%, a concentration range of the ethylenediamine tetraacetic acid is 0.01% to 10%, and a concentration range of the Accutase™ cell dissociation agent is 0.01× to 10×.

7. The method for extracting epidermal cells according to claim 1, wherein the epidermal cells are used for clinical research or treatment of burns, scalds, pigmentation and hypopigmentation, ulcers, aesthetic dermatology and the like.
